(19) United States
(12) Patent Application Publication
(10) Pub. No.: US 2016/0312168 A1
(43) Pub. Date: Oct. 27, 2016

(54) APPARATUS FOR CELL CULTIVATION

(71) Applicant: GE Healthcare Bio-Sciences Corp., Marlborough, MA (US)

(72) Inventor: Vincent Francis PIZZI, Westborough, MA (US)

(21) Appl. No.: 15/105,505

(22) PCT Filed: Dec. 29, 2014
(86) PCT No.: PCT/SE2014/051575

§ 371 Date: Jun. 16, 2016

Related U.S. Application Data
(60) Provisional application No. 61/983,482, filed on Apr. 24, 2014.
(60) Provisional application No. 61/921,626, filed on Dec. 30, 2013.

(51) Int. Cl. C12M 1/42; C12M 1/00; B01D 15/02; B01D 21/01; B01D 15/12; B01D 63/02; B01D 61/14; C12M 1/26; B01D 15/18

(52) U.S. Cl. CPC C12M 35/04; C12M 33/14; C12M 23/14; C12M 23/26; C12M 47/02; B01D 15/02; B01D 15/1807; B01D 15/1821; B01D 15/125; B01D 63/02; B01D 61/147; B01D 61/145; B01D 21/01

(57) ABSTRACT

The invention discloses an apparatus (1;11;31) for cell cultivation, comprising a bioreactor (2;12;32), an acoustic standing wave cell separator (5;15;35) and a filter (7;17;37), wherein an outlet (3;13;33) of the bioreactor is fluidically connected to an inlet (4;14;34) of the acoustic standing wave cell separator and a media outlet (6;16;36) of the acoustic standing wave cell separator is fluidically connected to the filter (7;17;37).

### TECHNICAL FIELD OF THE INVENTION

The present invention relates to cell cultivation, and more particularly to a bioreactor with an acoustic cell separation device and a filter. The invention also relates to methods of cultivating cells in such bioreactor systems.

### BACKGROUND OF THE INVENTION

In bioprocess settings, cells are cultivated in order to express proteins useful for manufacture of therapeutics and also in order to produce antigens, e.g. virus particles, for vaccine manufacturing. In both cases, the continuous drive towards improved process economy has led to demands for high cell densities during cultivation. One way of achieving high cell densities is to perform the cultivation in perfusion mode. In this operation, cells are retained in the bioreactor, and toxic metabolic by-products are continuously removed. Feed, containing nutrients is continually added. This operation is capable of achieving high cell densities and more importantly, the cells can be maintained in a highly productive state for weeks-months. This achieves much higher yields and reduces the size of the bioreactor necessary. It is also a useful technique for cultivating primary or other slow growing cells. Perfusion operations have tremendous potential for growing the large number of cells needed for human cell and genetic therapy applications.

A recent development in perfusion cultivation is the alternating tangential flow (ATF) method described in e.g. U.S. Pat. No. 6,544,424, U.S. Pat. No. 8,119,368 and U.S. Pat. No. 8,222,001, which are hereby incorporated by reference in their entirety. Here, part of the cell culture is removed from the bioreactor and passed through a hollow fiber cartridge to allow removal of metabolites and optionally expressed proteins through the hollow fiber walls. In order to avoid clogging of the fiber lumens with cells, the cell culture flow has to be alternated back and forth through the fibers. This decreases the efficiency of the filtration and at very high cell densities there will still be a risk of lumen blockage.

Accordingly there is a need for improved solutions that allow cultivation at high cell densities without blockage of filters.

### SUMMARY OF THE INVENTION

One aspect of the invention is to provide an apparatus allowing efficient cell cultivation at high cell densities. This is achieved with an apparatus as defined in claim 1.

One advantage is that cell damage can be minimized Further advantages are that clogging any of filters can be prevented and that the available filter area can be efficiently utilized.

Another aspect of the invention is to provide a cultivation method allowing efficient operation at high cell densities. This is achieved with a method as defined in the claims.

A third aspect of the invention is to provide an apparatus allowing efficient recovery of biomolecules from high cell density cell cultures. This is achieved with an apparatus as defined in the claims.

One advantage is that filters or centrifuges are not required upstream of the separation column(s). A further advantage is that the apparatus can easily be adapted to continuous processing.

A fourth aspect is to provide an efficient recovery method for biomolecules produced in high cell density cell cultures. This is achieved with a method as defined in the claims.

Further suitable embodiments of the invention are described in the dependent claims.

### DRAWINGS

FIG. 1 shows an apparatus according to the invention.

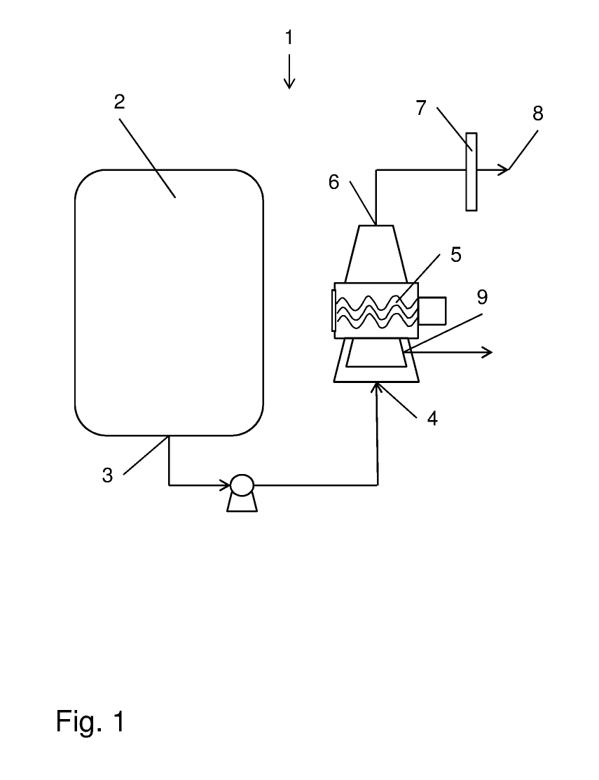

FIG. 2 shows an apparatus according to the invention with a crossflow filter device.

FIG. 3 shows an apparatus according to the invention with a hollow fiber cartridge.

FIG. 4 shows an apparatus according to the invention with a suction tube.

FIG. 5 shows two acoustic standing wave cell separators for use with the invention, a) with one acoustic resonation chamber and b) with two serially coupled acoustic resonation chambers.

FIG. 6 shows an apparatus according to the invention with three separation columns for alternating use.

FIG. 7 shows an apparatus according to the invention with two serially coupled separation columns.

### DETAILED DESCRIPTION OF EMBODIMENTS

In one aspect the present invention discloses an apparatus 1;11;31 for cell cultivation, comprising a bioreactor 2;12;32, an acoustic standing wave cell separator 5;15;35 and a filter 7;17;37. The acoustic standing wave cell separator can e.g. be a separator as described in U.S. Pat. No. 5,626,767, which is hereby incorporated by reference in its entirety. The separator can typically have an inlet 4;14;34 for the cell culture and a cell concentrate outlet 9;18;39 as well as a media outlet 6;16;36 for culture media depleted of cells. An outlet 3;13;33 of the bioreactor is fluidically connected to the inlet 4;14;34 of the acoustic standing wave cell separator and the media outlet 6;16;36 of the acoustic standing wave cell separator is fluidically connected to the filter 7;17;37. The bioreactor can be any type of bioreactor suitable for cell cultivation in 500 ml scale and larger (up to several m³). It can e.g. be a bioreactor comprising a flexible plastic bag, which can be supplied presterilized and used either on its own, such as in a rocking platform bioreactor of the WAVE type (GE Healthcare) or the flexible plastic bag can be used as an insert in a rigid support vessel such as in an Xcellerex XDR bioreactor (GE Healthcare). The fluidic connection between the bioreactor outlet and the inlet of the cell separator and/or between the outlet of the cell separator and the filter can e.g. be achieved by tubing, by direct connection or by some other type of conduit or structure amenable to transport of liquids. The connections may further comprise one or more pumps to convey the cell culture/culture media and optionally valves for controlling the flow.

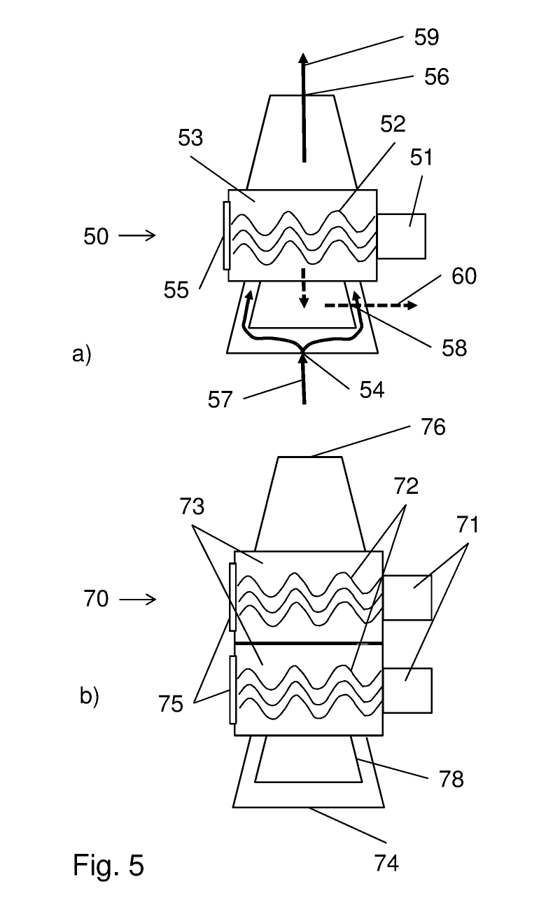

Examples of acoustic standing wave cell separators 50;70 for use in the invention are shown in FIG. 5 a) and b). They can contain one or more transducers 51;71, e.g. piezoelectric ultrasound transducers, adapted to generate an acoustic standing wave 52;72 in one or more acoustic resonation chambers 53;73. Each resonation chamber may also comprise an acoustic mirror 55;75 to stabilize the standing wave. When a cell suspension 57 is conveyed through the resonation chamber(s) via an inlet 54;74, cells are retained by the nodes of the standing waves, such that a cell-depleted liquid 59 can be obtained from the chamber(s) via a media outlet 56;76. By properly controlling the wave pattern it is also possible to withdraw a concentrate 60 enriched in cells via a cell concentrate outlet 58;78. FIG. 5 a) shows a separator 50 with a single resonation chamber 53, while FIG. 5 b) shows a separator 70 with two serially coupled resonation chambers 73, which is capable of further reducing the cell content in the stream from the media outlet 76. Suitable separators as described above are commercially available under the name of BioSep from Applikon Biotechnology (Netherlands). Typical reductions in cell density can be 98% or more when working at original cell densities of e.g. 100×10⁶ cells/ml in the feed to the separator.

The considerable depletion of cells obtainable by the acoustic cell separator means that even if a very high cell density is applied in the separator inlet, the cell depleted culture medium obtained in the media outlet has such a low density that the blockage of a filter applied afterwards is dramatically reduced. In practice this means that a normal flow filter (e.g. a depth filter) applied can be used several times longer without exchange and that crossflow filters can be used essentially without any blocking issues.

In some embodiments, the filter is a crossflow filter device 17 with a retentate side 20 and a permeate side 21. The media outlet 16 of the acoustic standing wave cell separator 15 can then be fluidically connected to an inlet 22 of the retentate side, while the cell concentrate outlet 18 of the acoustic standing wave cell separator and an outlet 23 of the retentate side can fluidically connected to an inlet 19 of the bioreactor. The apparatus can suitably be adapted to recover a permeate 24 from the permeate side, e.g. by having an outlet from the permeate side fluidically connected with a permeate recovery vessel or by feeding the permeate directly into a subsequent processing step. The fluidic connections between the media outlet and the retentate inlet, between the retentate outlet and the bioreactor inlet and/or between the cell concentrate outlet and the bioreactor inlet can e.g. be achieved by tubing, by direct connection or by some other type of conduit or structure amenable to transport of liquids. The connections may further comprise one or more pumps to convey the cell culture/culture media and optionally valves for controlling the flow.

The crossflow filter device can e.g. be a hollow fiber filter cartridge or it may alternatively be a flat sheet cassette device or plate-frame module. The crossflow filter device can suitably comprise a microfiltration membrane, e.g. with nominal pore size rating 0.1-5 micrometers, or an ultrafiltration membrane, e.g. with cutoff 10-500 kD. This setup allows for perfusion cultivation up to very high cell densities without any issues of filter/fiber blockage and there is no need for any pulsing or alternating flow in the filter device. A particular advantage of combining the acoustic separator with a crossflow filter device is that the acoustic separator provides a very gentle separation with minimal mechanical damage to fragile animal cells. As crossflow filtration involves high flow rates through narrow channels and the entries and exits of these channels, the risk of cell damage is much higher in the crossflow filtration (in particular at high cell densities) and by significantly reducing the cell density before application to the crossflow filter, the total extent of cell damage can be dramatically reduced. As damaged cells release cell debris, DNA and other potential foulants, this will improve the efficiency of both the crossflow filtration and any subsequent processing. Another advantage is that no alternating flow is needed to avoid blockage in the crossflow filter device, which means that the filter area is continuously being used for separation without any backward flushing cycles.

In certain embodiments, the outlet 33 is a suction tube adapted to withdraw a supernatant from the bioreactor 32. The suction tube may e.g. extend from the top side (during use) of the bioreactor downwards to a position in the lower half of the bioreactor, such as at a distance of 10-50% of the inner height of the bioreactor from the bottom of the bioreactor. The position of the suction tube may also be adjustable, e.g. by telescoping, to allow positioning of the tube end just above a cell sediment layer in the bioreactor. This enables withdrawal of a supernatant to the acoustic cell separator and subsequent filtering of the cell depleted supernatant through a filter, essentially without any filter blockage, even if a normal flow filter is used.

As discussed below, the apparatus can further comprise one or more separation columns fluidically connected to the filter. They are suitably arranged to receive a filtrate or permeate from the filter and can be either chromatography columns, such as packed bed chromatography columns, or expanded bed adsorption columns They can further be arranged for continuous or semi-continuous use, such as by simulated moving bed or periodic countercurrent chromatography. In this way a continuous process downstream of the bioreactor can be achieved.

In one aspect the present invention discloses a method of cultivating cells, comprising the steps of:
a) providing an apparatus 1;11;31 as described above;
b) introducing a culture medium and cells in said bioreactor 2;12;32;
c) cultivating cells in said bioreactor, and;
d) withdrawing a filtrate 8;38 or permeate 24 via said acoustic standing wave cell separator 5;15;35 and said filter 7;17;37.

The cells can e.g. be eukaryotic cells such as animal cells (e.g. mammalian, avian or insect cells) or fungal cells (e.g. mold or yeast cells). They can in particular be cells capable of expressing therapeutic biomolecules, such as immunoglobulins (e.g. monoclonal antibodies or antibody fragments), fusion proteins, coagulation factors, interferons, insulin, growth hormones or other recombinant proteins. Such cells can e.g. be CHO cells, Baby hamster kidney (BHK) cells, PER.C.6 cells, myeloma cells, HER cells etc. Suitably a small number of cells and a cell culture medium are introduced in the bioreactor and the cultivation conditions are selected such that the cells divide and thus produce an increasing cell density, while expressing the target biomolecule.

The cultivation can be performed according to methods known in the art, involving e.g. a suitable extent of agitation, addition of oxygen/air, removal of CO₂ and other gaseous metabolites etc. During cultivation, various parameters, such as e.g. pH, conductivity, metabolite concentrations, cell density etc. can be controlled to provide suitable conditions for the given cell type. The cell density can suitably be increased to a level where the cell concentration in the bioreactor during at least part of step c) (e.g. at the end of step c)) is at least 10×10⁶ cells/ml, such as at least 25×10⁶ cells/ml, 25-150×10⁶ or 50-120×10⁶ cells/ml. The upper limit will mainly be set by the rheological properties of the cell suspension at very high cell densities, where agitation and gas exchange can be hampered when paste-like consistencies are approached. The cell viability can e.g. be at least 50%, such as at least 80% or at least 90%. The concentration of a target biomolecule or target protein expressed by the cells can in the bioreactor during at least part of step c) (e.g. at the end of step c)), be at least 5 g/l or at least 10 g/l.

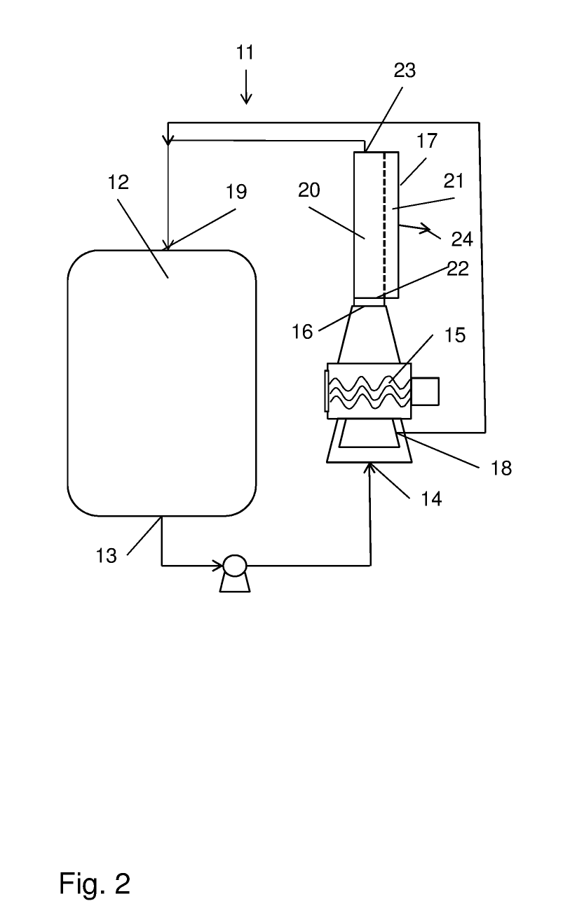

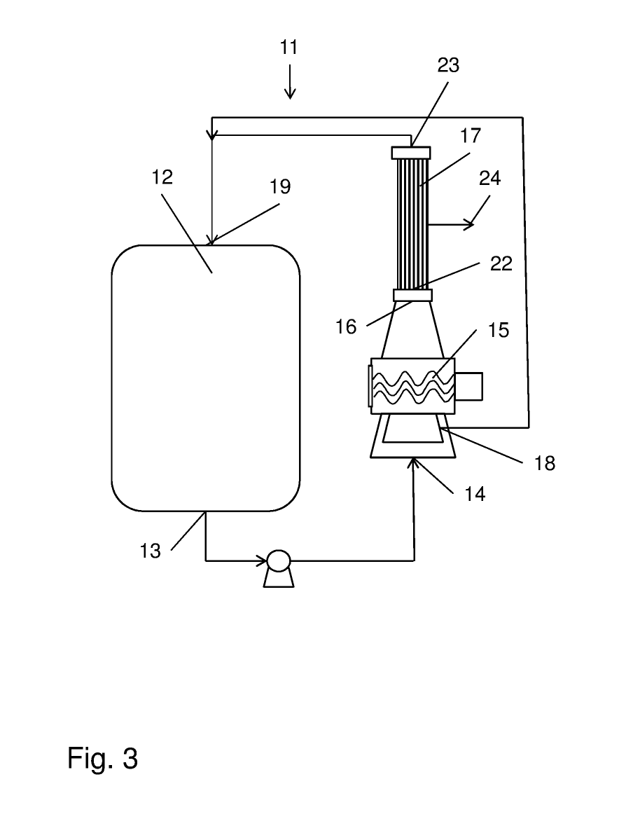

In certain embodiments illustrated by FIGS. 2 and 3, step a) comprises providing the apparatus 11 described above and step d) comprises withdrawing a permeate 24 and recycling both of i) a cell concentrate from said acoustic standing wave cell separator 15 and ii) a retentate from said crossflow filter device 17 to said bioreactor 12. Fresh culture medium can suitable be added to the bioreactor to compensate for the volume loss of the withdrawn permeate. If the crossflow filter device comprises a microfiltration membrane, the permeate will contain the expressed biomolecule which can be collected and further processed by e.g. one or more chromatography steps. It can e.g. be conveyed directly to an affinity chromatography column such as a protein A column if the biomolecule contains an Fc moiety (e.g. if it is an immunoglobulin or an immunoglobulin fusion protein). If the crossflow filter device comprises an ultrafiltration membrane, proteins will be retained while toxic and/or inhibiting metabolites will be removed. In this case, a target protein can be recovered after cultivation in a separate harvest operation.

As discussed above, the acoustic separator provides a gentle but efficient removal of cells such that cell-depleted culture medium can be fed into the inlet of the crossflow filter device without cell clogging or fouling issues. The cell concentrate from the acoustic separator can be fed back to the bioreactor for further culture, together with the retentate from the crossflow filter device.

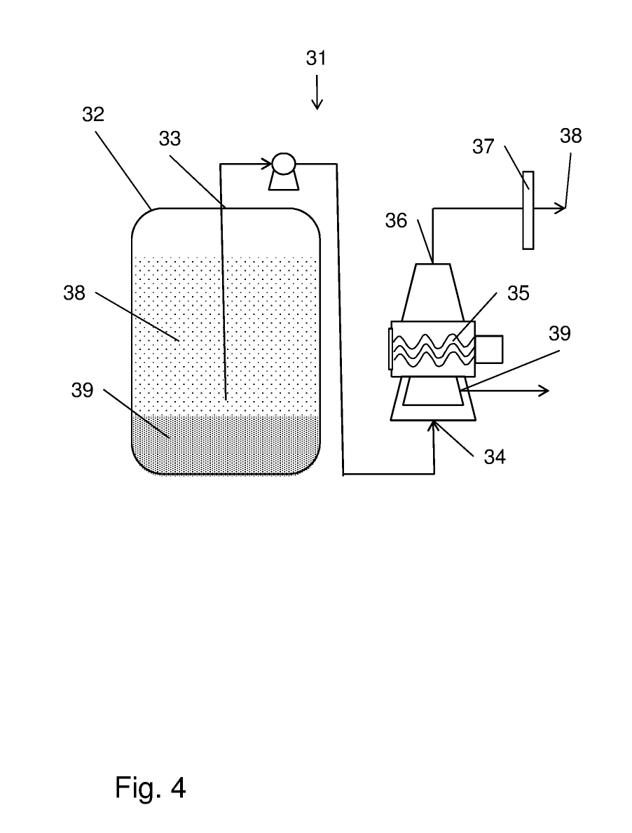

In some embodiments illustrated by FIG. 4, step a) comprises providing the apparatus 31 described above and wherein the method further comprises, before step d), a step c′) of adding a flocculant or precipitant to the bioreactor and allowing the formation of a supernatant and a sediment. The supernatant can then in step d) be withdrawn through suction tube 33 and delivered via the separator 35 to the filter 37. Individual cells sediment so slowly that it is impractical to separate them by gravity sedimentation. However, if they can be aggregated by addition of a flocculant, the sedimentation rate can be dramatically increased. The flocculant can e.g. be a soluble polymer such as chitosan, polyvinylpyridine or other polyelectrolytes. It can also be a multivalent salt, particularly in combination with particulates like calcium phosphates (e.g. hydroxyapatite as described in WO2007035283A1, which is hereby incorporated by reference in its entirety). Flocculants can also act as more or less selective precipitants for undesired cell culture components, e.g. host cell proteins. As the flocculated cells with any precipitated components sediment, a supernatant can be withdrawn via the acoustic cell separator to remove any non-sedimented cells and finally clarified by passage through a filter. An advantage of using the acoustic cell separator here is that the sedimentation does not have to be entirely complete, which saves time, and that a more complete withdrawal of supernatant can be performed (increasing the recovery of valuable target biomolecule) as the suction tube can be operated very close to the top of the sediment.

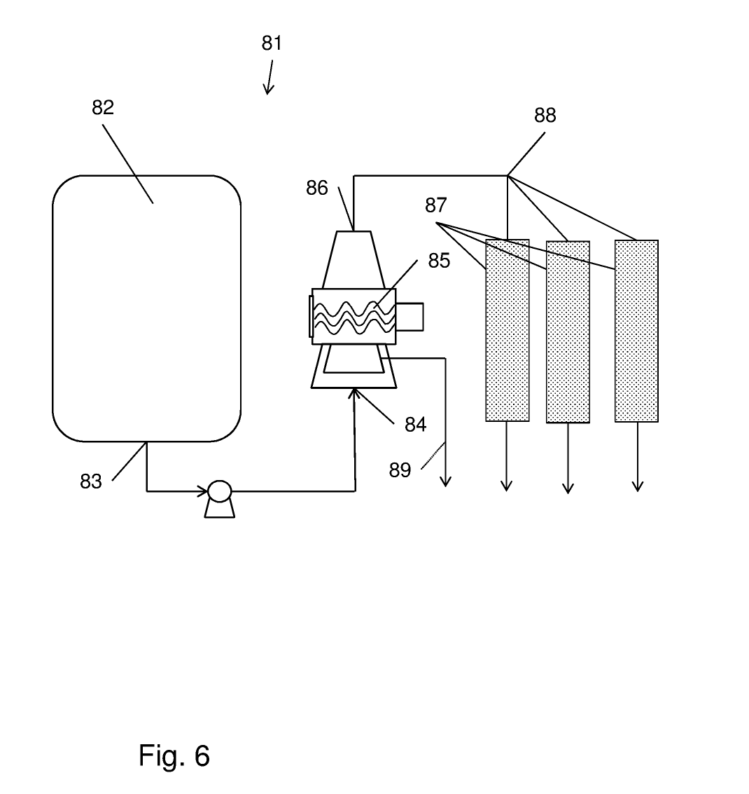

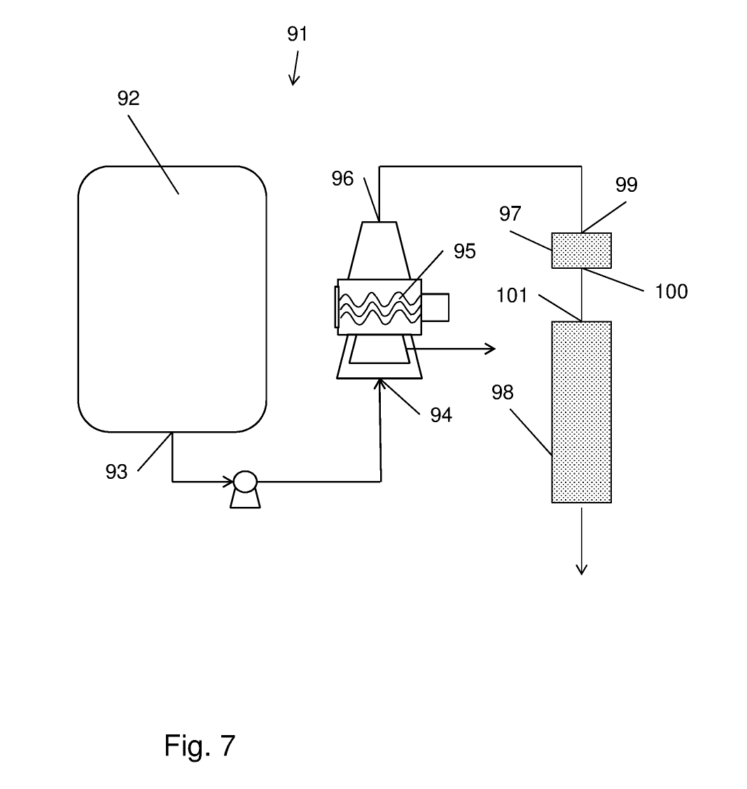

In a third aspect the invention discloses an apparatus 81;91 for recovery of biomolecules, as illustrated by FIGS. 6 and 7. The apparatus comprises a bioreactor 82;92 as discussed above, an acoustic standing wave cell separator 85;95 as discussed above and at least one separation column 87;97,98. In the apparatus, an outlet 83;93 of the bioreactor is fluidically connected to an inlet 84;94 of the acoustic standing wave cell separator 85;95 and a media outlet 86;96 of the acoustic standing wave cell separator is fluidically connected to the separation column(s) 87;97,98. The apparatus can optionally comprise a filter, as discussed above, between the media outlet and the separation column(s), but it can also be used without any filter as the cell depleted fraction obtainable from the media outlet has such a low cell concentration that it can be applied directly to a separation column. The media outlet may thus be directly connected to the separation column(s). The separation column(s) can suitably comprise a separation matrix capable of binding a target biomolecule produced in the bioreactor. If the biomolecule is an antibody or another Fc-containing protein, the separation matrix can e.g. be a protein A matrix such as the STREAMLINE™ rProtein A, MabSelect™ or MabSelect SuRe (GE Healthcare Life Sciences) matrices which bind Fc-containing proteins with high selectivity and allow the elution of highly purified antibodies/Fc-containing proteins. The separation column(s) can alternatively comprise other types of separation matrices such as e.g. ion exchange matrices, multimodal matrices or hydrophobic interaction matrices. If a plurality of columns 87 are used as indicated in FIG. 6, a valve 88 may allow sequential switching between the columns in order to switch to a fresh column when a previous one is becoming fully loaded. This concept can be further developed into continuous chromatography processes such as the simulated moving bed (SMB) or periodic counter-current (PCC) processes known in the art of chromatography, e.g. as described in U.S. Pat. No. 7,901,581, US20130213884 and US20120091063, which are hereby incorporated by reference in their entireties. The use of continuous chromatography in combination with the acoustic standing wave cell separator is particularly advantageous in that it allows all-continuous processing downstream of the bioreactor. If the cell-enriched concentrate 89 from the separator is recycled to the bioreactor, it is also possible to run all-continuous processing including the cell cultivation step.

In some embodiments, the separation column(s) comprise an expanded bed adsorption (EBA) column. This type of column comprises separation matrix particles of high density (typically 1.1-1.5 g/cm³) and the feed is applied to a bottom end of the column in an upwards direction such that the particle bed is expanded by the flow of the feed. In EBA feeds containing cells or other particles can be applied without immediate clogging of the column, as the interstices between the particles in the expanded bed are large enough to permit passage of the cells. However, there is a risk of cells attaching to the particle surfaces, causing fouling with time. When the cell concentration has been diminished by the passage through the standing acoustic wave cell separator, this risk can be avoided and fouling can easily be mitigated by cleaning of the matrix particles, e.g. with alkali such as 0.1-1 M NaOH, between cycles. Further details of EBA are provided in e.g. U.S. Pat. No. 5,522,993, U.S. Pat. No. 5,759,395, U.S. Pat. No. 5,866,006, U.S. Pat. No. 5,935,442, U.S. Pat. No. 6,325,937 and U.S. Pat. No. 6,620,326, which are hereby incorporated by reference in their entireties. Columns and matrices for EBA can e.g. be obtained from GE Healthcare Life Sciences or DSM Biologics under the trade names of STREAMLINE™ and Rhobust™ respectively.

In certain embodiments, at least one separation column comprises a packed bed of separation matrix particles. In order for the bed to have a low sensitivity towards any remaining cells or other particles, it can be advantageous if the separation matrix particles have a high (volume weighted) average diameter, such as at least 80 micrometers, at least 150 micrometers or at least 200 micrometers. The volume weighted average diameter can suitably be in the ranges of 80-300 micrometers, such as 150-300 or 150-250 micrometers to allow for both low sensitivity to particulates and for rapid mass transport. Examples of separation matrices in these ranges are the Protein A-functional crosslinked agarose beads MabSelect™ and MabSelect SuRe (85 micrometers), the crosslinked agarose beads Sepharose™ FastFlow (90 micrometers) and the crosslinked agarose beads Sepharose Big Beads (200 micrometers) (all GE Healthcare Life Sciences).

In some embodiments, illustrated by FIG. 7, an inlet 99 of a guard column 97 packed with separation matrix particles is fluidically connected to the media outlet 96 of the acoustic wave cell separator 95 and an outlet 100 of the guard column is fluidically connected to an inlet 101 of a main column 98 packed with separation matrix particles. The average diameters of the particles can suitably be as disclosed above and the guard column can e.g. be packed with the same type of matrix as the main column. If any remaining cells or other particulates tend to clog the columns, they will be caught in the guard column, which can easily be exchanged when needed, e.g. after a specified number of cycles or even after each cycle. The guard column can suitably be smaller than the main column, e.g. having less than 50%, such as less than 25% or less than 10% of the volume of the main column.

In a fourth aspect the invention discloses a method of recovering a biomolecule from a cell culture, comprising the steps of:
a) providing an apparatus 81;91 as discussed above;
b) introducing a culture medium and cells, as discussed above, in the bioreactor 82;92;
c) cultivating the cells in the bioreactor to form a cell culture;
d) withdrawing at least a portion of said cell culture to the acoustic standing wave cell separator 85;95;
e) separating cells from the cell culture in the acoustic standing wave cell separator to form a cell depleted fraction;
f) conveying the cell depleted fraction to the separation column(s) 87;97,98, where the biomolecule may bind to a separation matrix and be eluted in purified form through application of an eluent to the column(s). Alternatively, contaminants present in the cell culture may bind to the separation matrix and the biomolecule can be recovered in a flowthrough and/or wash fraction from the column(s).

This method allows for a highly efficient recovery of the biomolecule without complex centrifugation operations as are currently used.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An apparatus for cell cultivation, comprising a bioreactor, an acoustic standing wave cell separator and a filter, wherein an outlet of said bioreactor is fluidically connected to an inlet of said acoustic standing wave cell separator and a media outlet of said acoustic standing wave cell separator is fluidically connected to said filter.

2. The apparatus of claim 1, wherein said filter is a crossflow filter device having a retentate side and a permeate side and wherein said media outlet of said acoustic standing wave cell separator is fluidically connected to an inlet of said retentate side, a cell concentrate outlet of said acoustic standing wave cell separator and an outlet of said retentate side are fluidically connected to an inlet of said bioreactor and wherein said apparatus is adapted to recover a permeate from said permeate side.

3. The apparatus of claim 2, wherein said crossflow filter device is a hollow fiber filter cartridge.

4. The apparatus of claim 3, wherein said crossflow filter comprises a microfiltration membrane with nominal pore size rating 0.1-5 micrometers or an ultrafiltration membrane with cutoff 10-500 kD.

5. The apparatus of claim 1, wherein said filter is a depth filter.

6. The apparatus of claim 1, wherein said outlet is a suction tube adapted to withdraw a supernatant from said bioreactor.

7. The apparatus of claim 1, wherein said acoustic standing wave cell separator comprises at least two serially coupled separator chambers.

8. The apparatus of claim 1, wherein said bioreactor comprises a flexible bag.

9. The apparatus of claim 1, further comprising at least one separation column, fluidically connected to said filter and arranged to receive a filtrate or permeate from said filter.

10. A method of cultivating cells, comprising the steps of:
a) providing an apparatus of claim 1;
b) introducing a culture medium and cells in said bioreactor;
c) cultivating cells in said bioreactor, and;
d) withdrawing a filtrate or permeate via said acoustic standing wave cell separator and said filter.

11. The method of claim 10, wherein step a) comprises providing said apparatus and step d) comprises withdrawing a permeate and recycling both of i) a cell concentrate from said acoustic standing wave cell separator and ii) a retentate from said crossflow filter device to said bioreactor.

12. The method of claim 10, wherein step a) comprises providing said apparatus and wherein the method further comprises, before step d), a step c′) of adding a flocculant or precipitant to the bioreactor and allowing the formation of a supernatant and a sediment.

13. The method of claim 10, wherein the cell concentration in said bioreactor during at least part of step c) is at least 10×10⁶ cells/ml, such as at least 15×10⁶ cells/ml.

14. The method of claim 10, wherein in said bioreactor during at least part of step c), the concentration of a target protein expressed by said cells is at least 5 g/l.

15. An apparatus for recovery of biomolecules, comprising a bioreactor, an acoustic standing wave cell separator and at least one separation column, wherein an outlet of said bioreactor is fluidically connected to an inlet of said acoustic standing wave cell separator and a media outlet of said acoustic standing wave cell separator is fluidically connected to said at least one separation column.

16. The apparatus of claim 15, which does not comprise any filter between said media outlet and said at least one separation column.

17. The apparatus of claim 16, comprising a plurality of separation columns adapted for continuous separation, such as by a simulated moving bed or periodic counter-current process.

18. The apparatus of claim 15, wherein said at least one separation column is at least one expanded bed adsorption column.

19. The apparatus of claim 15, wherein said at least one separation column comprises a packed bed of separation matrix particles, such as particles of at least 80 micrometers volume-weighted average diameter.

20. The apparatus of claim 19, wherein an inlet of a guard column packed with separation matrix particles is fluidically connected to the media outlet of the acoustic wave cell separator and an outlet of the guard column is fluidically connected to an inlet of a main column packed with separation matrix particles.

21. A method of recovering a biomolecule from a cell culture, comprising the steps of:
a) providing the apparatus of claim 14;
b) introducing a culture medium and cells in said bioreactor;
c) cultivating cells in said bioreactor to form a cell culture;
d) withdrawing at least a portion of said cell culture to said acoustic standing wave cell separator;
e) separating cells from said cell culture in said acoustic standing wave cell separator to form a cell depleted fraction;
f) conveying said cell depleted fraction to said at least one separation column.
